SEQUENCE LISTING (42 sequences)

SEQ ID NO: 1 - DNA, 230 bp, Homo sapiens
aagacccttattgctgtcctggaggggctggggacttgtgtctgcggggatcagagcgcacagggagtgcacatatccaggcaccaggactagggctggagtgaggggggggtatttcaattaccttctattggtctcccttctctacactcttgtaataaaatgtctatttttaatgtttgtacacaacaatccttctattctagcctgcattgagcttgcatgcttgc

SEQ ID NO: 2 - DNA, 113 bp, Homo sapiens
cgctgtgaacatgtccaggcgcggcgcttctccatcaattgaagaagacaccaccattctgaggccagggagcgagtgggggccagccacggggactcagcccttgtttctct

SEQ ID NO: 3 - DNA, 352 bp, Homo sapiens
taatcagaaaccctcattgtgtttctaccggagagagaatgtatttgctgacaaccattaaagtcagaagttttactccaggttattgcaataaagtataatgtttattaaatgcttcatttgtatgtcaaagctttgactctataagcaaattgcttttttccaaaacaaaaagatgtctcaggtttgttttgtgaattttctaaaagctttcatgtcccagaacttagcctttacctgtgaagtgttactacagccttaatattttcctagtagatctatattagatcaaatagttgcatagcagtatatgttaatttgtgtgtttttagctgtgacacaactgtgtgat

SEQ ID NO: 4 - DNA, 133 bp, Homo sapiens
gggagaggggcgcagctaagactgggggcaggatggcggaaaggaaggggcgtggtggctagagggaagagaagaaacagaaggggctcagttcaccttgataaggtgcttccgagctgggatgagaggaagg

SEQ ID NO: 5 - DNA, 164 bp, Homo sapiens
cagtggtgccatctctgtccactgcaacctccacatcctgggttcaagtgattctcctgcctcagcctctggaggagctggtatcacaggcgtcccccaccacgcctggctaaattttgtatttttaggtggtcttgaactcctgatgtcaggtgattctccta

SEQ ID NO: 6 - DNA, 100 bp, Homo sapiens
agaatgctagcccatctggctgctgatctgctatcacctgcaactctttttttatcaaggacattcccactattatgtcttctctgacctctcatcaaat

SEQ ID NO: 7 - DNA, 215 bp, Homo sapiens
ttggaggtctgaatttggaaaaaaaaactatgtccaggagcagctgtaacctgtagggaaataatggaacaatcatccataagagggatgaacattaagtgtttgaattcatgctctgcttttgtgttactgtaaacacaagatcaagatttggataatctttttcctttgtgtttccaacttagatcatgtctaaatatatgctttcatatggc

SEQ ID NO: 8 - DNA, 68 bp, Homo sapiens
tttgtgtattataattcaaaggcatttcttttctgcatgttctaaataaaaagcattattatttgctg

SEQ ID NO: 9 - DNA, 146 bp, Homo sapiens
tttcactatctgtgatgcttcttctgacccgtcatctcacattttcccttcccccaagatctagtgaacattcagcctccattaaaaaagtttcactgtgcaaatatatctgctattccccatactctataatagttacattgagt

SEQ ID NO: 10 - DNA, 70 bp, Homo sapiens
ggggtacctagtccccagcctgctcctagcccagaggctctaatgtacaataaagcaatgtggtagttcc

SEQ ID NO: 11 - DNA, 79 bp, Homo sapiens
cgctttcaaacaagttttcaaattgtttgaggtcaggatttctcaaactgattcctttctttgcatatgagtatttgaa

SEQ ID NO: 12 - DNA, 290 bp, Homo sapiens
cgggcttcatccaatggactgcataaataaccggggattctgtacatgcattgagctctctcattgtctgtgtagagtgttatacttgggaatataaaggaggtgaccaaatcagtgtgaggaggtagatttggctcctttgcttctcacgggactatttccaccacccccagttagcaccattaactcctcctgagctctgataagagaatcaacatttctcaataatttcctccacaaattattaatgaaaataagaattattttgatggctctaacaatgacattta

SEQ ID NO: 13 - DNA, 88 bp, Homo sapiens
ttggtcttcaagaaagctgtgccccagaacaccagagatttcaacttagtcaataaaaccttgaaataaagatgggcttaatctaatg

SEQ ID NO: 14 - DNA, 304 bp, Homo sapiens
tctctcatggtctgtatagagtgttatacttggtaatatagaggagatgaccaaatcagtgctggggaagtagatttggcttctctgcttctcataggactatctccaccacccccagttagcaccattaactcctcctgagctctgataacataattaacatttctcaataatttcaaccacaatcattaataaaaataggaattattttgatggctctaacagtgacatttatatcatgtgttatatctgtagtattctatagtaagctttatattaagcaaatcaataaaaacctctttac

SEQ ID NO: 15 - DNA, 19 bp, Homo sapiens
aagacccttattgctgtcc

SEQ ID NO: 16 - DNA, 19 bp, Homo sapiens
gcaagcatgcaagctcaat

SEQ ID NO: 17 - DNA, 19 bp, Homo sapiens
cgctgtgaacatgtccagg

SEQ ID NO: 18 - DNA, 20 bp, Homo sapiens
agagaaacaagggctgagtc

SEQ ID NO: 19 - DNA, 21 bp, Homo sapiens
taatcagaaaccctcattgtg

SEQ ID NO: 20 - DNA, 20 bp, Homo sapiens
atcacacagttgtgtcacag

SEQ ID NO: 21 - DNA, 19 bp, Homo sapiens
gggagaggggcgcagctaa

SEQ ID NO: 22 - DNA, 18 bp, Homo sapiens
ccttcctctcatcccagc

SEQ ID NO: 23 - DNA, 23 bp, Homo sapiens
cagtggtgccatctctgtccact

SEQ ID NO: 24 - DNA, 21 bp, Homo sapiens
tagggagaatcacctgacatc

SEQ ID NO: 25 - DNA, 16 bp, Homo sapiens
agaatgctagcccatc

SEQ ID NO: 26 - DNA, 19 bp, Homo sapiens
atttgatgagaggtcagag

SEQ ID NO: 27 - DNA, 20 bp, Homo sapiens
ttggaggtctgaatttggaa

SEQ ID NO: 28 - DNA, 16 bp, Homo sapiens
gccatatgaaagcata

SEQ ID NO: 29 - DNA, 17 bp, Homo sapiens
tttgtgtattataattc

SEQ ID NO: 30 - DNA, 16 bp, Homo sapiens
cagcaaataataatgc

SEQ ID NO: 31 - DNA, 21 bp, Homo sapiens
tttcactatctgtgatgcttc

SEQ ID NO: 32 - DNA, 26 bp, Homo sapiens
actcaatgtaactattatagagtatg

SEQ ID NO: 33 - DNA, 16 bp, Homo sapiens
ggggtacctagtcccc

SEQ ID NO: 34 - DNA, 21 bp, Homo sapiens
ggaactaccacattgctttat

SEQ ID NO: 35 - DNA, 21 bp, Homo sapiens
cgctttcaaacaagttttcaa

SEQ ID NO: 36 - DNA, 21 bp, Homo sapiens
ttcaaatactcatatgcaaag

SEQ ID NO: 37 - DNA, 20 bp, Homo sapiens
cgggcttcatccaatggact

SEQ ID NO: 38 - DNA, 23 bp, Homo sapiens
taaatgtcattgttagagccatc

SEQ ID NO: 39 - DNA, 23 bp, Homo sapiens
tggtcttcaagaaagctgtgccc

SEQ ID NO: 40 - DNA, 21 bp, Homo sapiens
cattagattaagcccatcttt

SEQ ID NO: 41 - DNA, 20 bp, Homo sapiens
ctcatggtctgtatagagtg

SEQ ID NO: 42 - DNA, 23 bp, Homo sapiens
gtaaagaggtttttattgatttg

(19) United States
(12) Patent Application Publication
(10) Pub. No.: US 2004/0171015 A1
(43) Pub. Date: Sep. 2, 2004

(54) Cyp450-specific dna probes and primers, and biological applications thereof

(76) Inventors: Isabelle De Waziers, Paris (FR); Corinne Couteau, Maisons-Alfort (FR); Celine Gros, Tremblay-En (FR); Arlette Moncion, Paris (FR); Philippe Beaune, Paris (FR)

Correspondence Address: NIXON & VANDERHYE, PC, 1100 N GLEBE ROAD, 8TH FLOOR, ARLINGTON, VA, 22201-4714, US

(21) Appl. No.: 10/480,276

(22) PCT Filed: Jun. 11, 2001
(86) PCT No.: PCT/EP01/07056

(51) Int. Cl. C12Q 1/68; C07H 21/04; C12P 19/34

(52) U.S. Cl. 435/6; 435/91.2; 536/24.3

(57) ABSTRACT

The present application relates to a set of new cDNA probes which enables the specific and simultaneous detection of the main fourteen CYP450 isoforms, and to new primers specific to the probes. It also provides transfection vectors, genetically engineered cells and with amplification products derived therefrom. The present application notably encompasses any solid surface such as DNA filters or DNA chips which comprises a cDNA probe or an amplification product of the invention. The products provided thereby enables to evaluate the toxicity or pathogenicity of a product, and to predict drug in vivo interactions or efficiency.

### FIELD OF THE INVENTION

The present application relates to CYP450-specific DNA probes and primers, and to biological applications thereof The present application also relates to products derived from said probes and primers, namely transfection vectors carrying CYP450-specific DNA, cells transfected therewith, amplification products obtainable with said primers, DNA arrays comprising such CYP-specific cDNA probes, and to methods wherein such products are used.

### BACKGROUND OF THE INVENTION

CYP450 (CYtochromes P450) is a superfamily of enzymes which are located on the membrane of the endoplasmic reticulum. Their main function is to catalyze an oxygenation reaction in the presence of NADPH, an electron transporter (NADPH cytochrome P450 reductase) and molecular oxygen. Different CYP450 belong to the same family (symbolized by an arabic number) when their amino acid sequence similarity is superior to 40%, and to the same sub-family (symbolized by an upper case letter) when their amino acid sequence similarity is above 55%. Up to now, three CYP450 families have been identified as involved in xenobiotics metabolism (families CYP 1, CYP 2 and CYP 3). CYP 1 family is known to comprise two sub-families: sub-family CYP 1A (with isoforms CYP 1A1 and CYP 1A2) and CYP 1B (with isoform CYP 1B1). CYP 2 family is known to comprise five sub-families: sub-family CYP 2A (with isoforms CYP 2A6, CYP 2A7, CYP 2A13), sub-family CYP 2B (with isoform CYP 2B6), sub-family 2C (with isoforms CYP 2C8, CYP 2C9, CYP 2C 18, CYP 2C19), sub-family 2D (with isoform CYP 2D6), and sub-family CYP 2E (with isoform CYP 2E1). Family 3 comprises sub-family CYP 3A with isoforms CYP 3A4, CYP 3A5 and CYP 3A7.

Some CYP450 metabolize endogenous substrates (such as steroids, fatty acids, prostaglandins), others metabolize xenobiotics (i.e. low molecular weight molecules such as drugs, smoke compounds (especially cigarette smoke compounds), atmospheric pollutants, compounds of food origin). CYP450 expression pattern thus mirrors its metabolic abilities or deficiencies of the organism.

But the expression of CYP450 is highly variable: there are a tissue variability, a physio-pathological variability, a genetic variability, and an environmental variability. Indeed, whereas almost all human tissues express some CYP450 isoforms, most isoforms are located in the main organ for detoxification, i.e. in the liver, and also in those tissues which have exchanges with the environment (small intestine, lungs, kidneys, skin). CYP450 are mainly located in the hepatocyte cells, but all cellular types of the same tissue do not express a CYP450, and their respective CYP450 cellular compositions differ from each other. Some CYP450 are mainly hepatic (e.g. CYP 1A2 and CYP 2C9), others are more extra-hepatic (e.g. CYP 1A1). In addition, there is a physiological variability, i.e. some physiological parameters such age, alimentation pattern, pathological conditions have an influence on CYP450 expression pattern. For example, CYP 3A7 is strongly expressed in fetal liver whereas in adult liver it is only expressed at low concentrations. Alimentation pattern style also alters CYP 450 expression pattern (e.g. crucifers—such as cabbage—, grapefruit juice, carbohydrate-rich food, or starvation). Furthermore, pathological conditions such as diabetes, hepatitis, cirrhosis alter CYP450 expression pattern in such a way that the organism has reduced metabolic capacities. Besides, there is also a genetic variability. Some CYP450 have genetic polymorphism which can alter their expression or their catalytic activities. For example, it has been demonstrated that CYP 2D6 polymorphisms (CYP 2D6 metabolizes debrisoquine, sparteine and dextrometorphane) is directly linked with the different phenotypes observed (slow metabolism/fast metabolism/ultra-fast metabolism). CYP450 expression pattern is furthermore influenced by environmental factors: xenobiotics such as smoke (especially cigarette smoke), food, drugs modify the expression pattern, and thereby lead to a metabolism that is either induced, repressed or inhibited.

Enzymatic induction is an adaptative response which stimulates the elimination of xenobiotics and thereby protects cells against them. For example, exposure to aromatic polycyclic hydrocarbons (contained in the smoke of cigarettes), such as benzo[a]pyrene, induces an increase in CYP 1A1 and CYP 1A2 tissue concentrations. Administration of rifampicin or of phenobarbital induces the expression of CYP 3A4 in humans. Induction of CYP450 is mainly due to transcriptional activation. On the other hand, CYP450 repression can also occur, notably pursuant to exposure to xenobiotics such as cytokines (e.g. interferon) which induces a decrease in stable mRNA synthesis. There may also be a CYP450 inhibition: it may be due either to (competitive or non-competitive) reversible inhibitions, or to irreversible inhibitions. Reversible inhibition stops when the inhibiting substrate is no longer administered (the inhibiting substrate binds to the active site thus impeding another substrate from linking thereto, or it binds to another interfering site). In the case of irreversible inhibitions however, the inhibiting substrate (also called suicide substrate) links to the active site where it is transformed into a reactant metabolite which in turn binds to the CYP450 and permanently inactivates it. In this latter case, the enzymatic activity will only be restored when new CYP450 molecules are synthesized. First generation macrolides such as troleandomycine, erythromycine, or some steroids such as ethinyleostradiol, gestodene bind to the heme group. Others, such as chloramphenicol, bind to amino acids which are essential to the catalytic activity of the enzyme. Among non-drug xenobiotics, 6′,7′dihydroxybergamottin, which is the main furanocoumarine in grapefruit juice, inhibits CYP 3A4 by catalytic destruction.

Concomitant administration of two or more drugs can thus have deleterious effects on their respective pharmaco-kinetics, and more particularly on their metabolisms. Such variations involve the afore-mentioned mechanisms of CYP450 induction and/or repression and/or inhibition. The efficiency or the toxicity of a drug is hence closely linked to the CYP450 expression pattern it induces. Table 1 below gives an illustration of some known drug interactions.

TABLE 1
some examples of drug interactions

| | Drug responsible | | | | |
|---|---|---|---|---|---|
| Type of | for the | Concerned | | | |
| interaction | interaction | drug | Effect | Clinical consequences | CYP |
| Induction | secobarbital | warfarine | decrease or | no anti-coagulating effect: | CYP 2C9 |
| | | | lost of | danger of thrombo- | |
| | | | efficiency | embolic disease | |
| | ethanol (chronic | paracetamol | | danger of hepatotoxicity | CYP 2E1 |
| | administration) | | | | |
| | rifampicine- | oral | | danger of pregnancy | CYP 3A4 |
| | griseofutvine | contraceptive | | | |
| | rifampicine | cyclosporine A | | danger of graft rejection | CYP 3A4 |
| Repression | IFNα, IFNβ | theophylline | decrease in | danger of asthma | CYP 1A2 |
| | | | efficiency | | |
| Inhibition | fluconazole | NSAID | increase in | haemorrhage and gastro | CYP 2C9 |
| | | (ibuprofene, | toxicity and/or | intestinal perforations | |
| | | piroxicam, | in undesired | | |
| | | diclofenac) | side-effects | | |
| | cimetidine | imipramine | | atropinical effects, | CYP 2D6 |
| | | | | cardiovascular toxicity | |
| | ketoconazole, | triazolam | | sleep-wakefulness | CYP 3A4 |
| | itraconazole | | | alternation, coma, | |
| | | | | respiratory depression | |
| | erythromycine, | cisapride, | | | CYP 3A4 |
| | ketoconazole | terfenadine | | | |
| | verapamil, | simvastatine, | | rhabdomyolysis | CYP 3A4 |
| | diltiazem, | lovastatine | | | |
| | erythromycine | | | | |
| | itraconazole | felodipine | | hypotension, bradycardy | CYP 3A4 |
| | ritonavir | ergotamine | | ergotisme | CYP 3A4 |
| | erythromycine | carbamazepine | | ataxy, sleep-wakefulness | CYP 3A4 |
| | | | | alternation, | |
| | ketoconazole, | cyclosporine | | nephrotoxicity | CYP 3A4 |
| | erythromicine | | | | |
| | quinidine | codeine | | decrease in antalgic effect | CYP 2D6 |

NSAID: Non-Steroid Anti-Inflammatory Drugs

While the first concern is of course to avoid deleterious effects when several drugs are concomitantly administered, it has also to be kept in mind that drug interactions may also in some case be beneficial to the patient. For example, the anti-protease saquinavir has a very low in vivo efficiency due to its low bio-availability, but the concomitant administration of grapefruit juice or of ritonavir significantly increases its bio-availability, and thereby the efficiency of the saquinavir drug. Cyclosporine, which a very useful but also very expensive immuno-suppressor, is another example of such beneficial interactions: it has indeed been demonstrated that the concomitant administration of ketonazole doubles its bio-availability, thereby leading to a 60-80% decrease in the doses necessary to achieve actual immuno-suppression.

It is thus clear that CYP 450 expression pattern is a major indicator of how a compound will be metabolized by an organism or microorganism, and is a very valuable tool for evaluating the toxicity or pathogenicity of a product, for predicting (deleterious or beneficial) drug interactions, or drug in vivo efficiency. Only for a few compounds however is CYP450 expression pattern known. This is due to the fact that appropriate means for evaluating global CYP450 expression pattern are lacking at the present time. Indeed, most available tools (mainly, antibodies) target one isoform or the other without the required specificity, and none of them enables the simultaneous detection of the fourteen main human liver CYP450 isoforms, namely CYP 1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5, CYP 3A7.

### SUMMARY OF THE INVENTION

The present invention provides such a valuable and useful tool. The invention indeed provides a set of new 50-350 base length cDNA probes which altogether enable specific detection of each one of the fourteen main CYP450 isoforms.

The set of cDNA probes of the invention is a functional unity: it enables to simultaneously and specifically target the whole set of said fourteen main human liver CYP450, ie. the main fourteen human CYP450 whose expression can be altered by xenobiotics. Furthermore, the cDNA probes of the invention also share the common technical feature of not leading to any cross-hybridization (as evaluated by Northern blots) when incubated with total RNA from human hepatocytes: the cDNA probes of the invention, when taken as a whole, share a full specificity, and it is this unifying feature that makes them highly valuable tools for industrial application, i.e. single-step CYP450 detection and transcription assessment. To the best of the applicant's knowledge, this is the first time that such a probe set is made available.

Every cDNA probe of the invention has been isolated and purified from human genomic DNA. Each of them has the further advantage of being specifically isolable by polymerase chain reactions with specific primers. The cDNA probes of the invention have indeed such a sequence that primers with comparable Tm and actual specificity can be designed. The primers of the invention specifically frame the cDNA probes of the invention, thereby allowing their specific amplification and isolation from natural resources. The invention thus also provides new primer pairs with industrial utility.

The invention also provides transfection vectors comprising a cDNA probe of the invention, and genetically engineered cells transfected with such a transfection vector or with such a cDNA probe. It notably provides cells that produce said cDNA probes via DNA replication. The invention also relates to amplification products obtainable with the primers of the invention.

The cDNA probes and the amplification products of the invention are advantageously placed onto a solid surface so as to allow a simultaneous and specific detection of all or several of said CYP450. The invention particularly encompasses such solid surfaces as DNA-type filters (e.g. nylon® membrane) and arrays or micro-arrays such as DNA chips.

The provision of such specific CYP450 products enables CYP450 detection or transcription assessment with high specificity, high velocity (simultaneous detection of fourteen CYP450 isoforms), and low demands in terms of input biological sample. Such methods are encompassed by the present application, they are particularly useful for evaluating the toxicity or pathogenicity of a product, and for predicting drug in vivo interactions or efficiency.

### DETAILED DESCRIPTION OF THE INVENTION

The present invention relates to a group of cDNA probes which consists in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and the complementary sequences thereof. The present application encompasses every isolated and purified polynucleotide of this group.

SEQ ID NO: 1 is a DNA molecule which hybridizes to CYP 1A1,

SEQ ID NO: 2 is a DNA molecule which hybridizes to CYP 1A2,

SEQ ID NO: 3 is a DNA molecule which hybridizes to CYP 1B1,

SEQ ID NO: 4 is a DNA molecule which hybridizes to CYP 2A,

SEQ ID NO: 5 is a DNA molecule which hybridizes to CYP 2B6,

SEQ ID NO: 6 is a DNA molecule which hybridizes to CYP 2C8,

SEQ ID NO: 7 is a DNA molecule which hybridizes to CYP 2C18,

SEQ ID NO: 8 is a DNA molecule which hybridizes to CYP 2C9,

SEQ ID NO: 9 is a DNA molecule which hybridizes to CYP 2C19,

SEQ ID NO: 10 is a DNA molecule which hybridizes to CYP 2D6,

SEQ ID NO: 11 is a DNA molecule which hybridizes to CYP 2E 1,

SEQ ID NO: 12 is a DNA molecule which hybridizes to CYP 3A4,

SEQ ID NO: 13 is a DNA molecule which hybridizes to CYP 3A5,

SEQ ID NO: 14 is a DNA molecule which hybridizes to CYP 3A7.

Each isolated and purified polynucleotide of said group is of 50-350 base length, and has such a sequence that primers of comparable Tm and of actual specificity can be produced. The whole group of isolated and purified polynucleotides also has a functional unity as it enables the simultaneous and specific detection and/or transcription assessment of all their respective targets (the main fourteen human liver CYP450) without cross-hybridizing with any other (non-CYP450) total RNA reverse transcript from human hepatocyte. All isolated and purified polynucleotides of said group therefore share structural and functional features which make them altogether a valuable tool for simultaneous and specific detection and/or transcription assessment of the main fourteen human liver CYP450 isoforms.

As a further characteristic, each polynucleotide of said group hybridizes with its target without cross-hybridization with another polynucleotide of this group, nor with a CYP450 coding region. Moreover, each polynucleotide of said group, except SEQ ID NO: 9, hybridizes to its target CYP450 isoform without hybridizing to another CYP450 isoform. In addition to its target CYP450 isoform (Le. CYP 2C19), SEQ ID NO: 9 may also hybridize in the 3′-NTR of another CYP450 isoform, namely CYP 2C9 (SEQ ID NO: 8 however hybridizes its CYP 2C9 target without hybridizing to CYP 2C19). Differential detection with SEQ ID NO: 9 and NO: 8 is therefore preferably advised when it is desired to detect CYP 2C19 fully specifically.

As a preferred sub-group, the present application encompasses the sub-group consisting of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and the complementary sequences thereof (i.e. the polynucleotides which target CYP 1B1 and CYP450 family 2).

The present application also encompasses any one of said polynucleotides which is physically or chemically associated with a detection molecule (e.g. a radio-active label such as ³²P, or a fluorescent label).

The present invention also provides a group of primer pairs consisting of:

SEQ ID NO: 15 and SEQ ID NO: 16,

SEQ ID NO: 17 and SEQ ID NO: 18,

SEQ ID NO: 19 and SEQ ID NO: 20,

SEQ ID NO: 21 and SEQ ID NO: 22,

SEQ ID NO: 23 and SEQ ID NO: 24,

SEQ ID NO: 25 and SEQ ID NO: 26,

SEQ ID NO: 27 and SEQ ID NO: 28,

SEQ ID NO: 29 and SEQ ID NO: 30,

SEQ ID NO: 31 and SEQ ID NO: 32,

SEQ ID NO: 33 and SEQ ID NO: 34,

SEQ ID NO: 35 and SEQ ID NO: 36,

SEQ ID NO: 37 and SEQ ID NO: 38,

SEQ ID NO: 39 and SEQ ID NO: 40,

SEQ ID NO: 41 and SEQ ID NO: 42 and

their respective complementary oligonucleotide couples.

The present application encompasses every individual primer and primer pair from this group. As above-mentioned, the primers of each pair have comparable Tm, and are thus valuable PCR tools (standard PCR or RT-PCT, as well as quantitative PCR or RT-PCR).

When placed into contact with (reverse-transcribed) total RNA from human hepatocytes, primer pair SEQ ID NO: 15 (or its complementary sequence) and SEQ ID NO: 16 (or its complementary sequence) specifically frame SEQ ID NO: 1, and thereby enable SEQ ID NO: 1 specific amplification by standard polymerase chain reactions. Examples of such PCRs are given in the below examples. The same applies mutatis mutatandis to:

SEQ ID NO: 17 (or its complementary sequence) and SEQ ID NO: 18 (or its complementary sequence) primer pair, and the polynucleotide SEQ ID NO: 2,

SEQ ID NO: 19 (or its complementary sequence) and SEQ ID NO: 20 (or its complementary sequence) primer pair, and the polynucleotide SEQ ID NO: 3,

SEQ ID NO: 21 (or its complementary sequence) and SEQ ID NO: 22 (or its complementary sequence) primer pair, and the polynucleotide SEQ ID NO: 4,

SEQ ID NO: 23 (or its complementary sequence) and SEQ ID NO: 24 (or its complementary sequence) primer pair, and the polynucleotide SEQ ID NO: 5,

SEQ ID NO: 25 (or its complementary sequence) and SEQ ID NO: 26 (or its complementary sequence) primer pair, and the polynucleotide SEQ ID NO: 6,

SEQ ID NO: 27 (or its complementary sequence) and SEQ ID NO: 28 (or its complementary sequence) primer pair, and the polynucleotide SEQ ID NO: 7,

SEQ ID NO: 29 (or its complementary sequence) and SEQ ID NO: 30 (or its complementary sequence) primer pair, and the polynucleotide SEQ ID NO: 8,

SEQ ID NO: 31 (or its complementary sequence) and SEQ ID NO: 32 (or its complementary sequence) primer pair, and the polynucleotide SEQ ID NO: 9,

SEQ ID NO: 33 (or its complementary sequence) and SEQ ID NO: 34 (or its complementary sequence) primer pair, and the polynucleotide SEQ ID NO: 10,

SEQ ID NO: 35 (or its complementary sequence) and SEQ ID NO: 36 (or its complementary sequence) primer pair, and the polynucleotide SEQ ID NO: 11,

SEQ ID NO: 37 (or its complementary sequence) and SEQ ID NO: 38 (or its complementary sequence) primer pair, and the polynucleotide SEQ ID NO: 12,

SEQ ID NO: 39 (or its complementary sequence) and SEQ ID NO: 40 (or its complementary sequence) primer pair, and the polynucleotide SEQ ID NO: 13,

SEQ ID NO: 41 (or its complementary sequence) and SEQ ID NO: 42 (or its complementary sequence) primer pair, and the polynucleotide SEQ ID NO: 14.

With the primer pairs of the invention, new amplification products have been produced by polymerase chain reactions. Starting material can be any material which contains a CYP450 isoform selected from the group consisting of CYP 1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5, CYP 3A7. Examples of such starting material include any material of human, animal, plant or protist origin. Preferred starting material includes human liver, and human hepatocytes in particular.

Any PCR conditions which are considered as appropriate by a skilled person are convenient when said primer pairs are used as PCR primers. Standard PCR conditions and guidelines can e.g. be found in “PCR protocols, Current Methods and Applications” Ed. Bruce A. White. Preferred conditions for each primer pair are described in the examples below (namely example 1).

The present application hence encompasses any amplification product, obtainable by submitting a set of polynucleotides of human, animal, plant, or protist origin to polymerase chain reactions with at least one primer pair of the invention.

Transfection vectors which comprise a polynucleotide of the invention, and genetically engineered cells which have been transfected with such a polynucleotide or transfection vector have also been produced (see the examples given below). More particularly, the invention provides transfection vectors such as plasmids, cosmids, onto which a polynucleotide of the invention has been inserted, and which can replicate when inserted into a growing cell. Such transfection vectors and genetically engineered cells are encompassed by the present application. The genetically engineered cells inside which a polynucleotide of the invention can replicate when the cell is made to grow are appropriate tools for producing this polynucleotide, as the replicated polynucleotide can be purified from the cell by the skilled person following standard procedures in the field (see the examples below).

For easy detection and identification of the different CYP450 isoforms concerned, the polynucleotides, the amplification products, the transfection vectors and the genetically engineered cells of the invention are advantageously placed in such a manner that one can distinguish them. Solid surfaces are examples of such appropriate means for said polynucleotides. Multi-well PCT or RT-PCR are examples of such appropriate means when several of said primer pairs are used.

The present invention hence encompasses any solid surface (or carrier) which comprises at least one polynucleotide of the invention, or at least one amplification product of the invention, or at least one transfection vector of the invention, or at least one genetically engineered cell of the invention. It more particularly encompasses any solid surface (or carrier), which comprises:

SEQ ID NO: 1 or its complementary sequence,

SEQ ID NO: 2 or its complementary sequence,

SEQ ID NO: 3 or its complementary sequence,

SEQ ID NO: 4 or its complementary sequence,

SEQ ID NO: 5 or its complementary sequence,

SEQ ID NO: 6 or its complementary sequence,

SEQ ID NO: 7 or its complementary sequence,

SEQ ID NO: 8 or its complementary sequence,

SEQ ID NO: 9 or its complementary sequence,

SEQ ID NO: 10 or its complementary sequence,

SEQ ID NO: 11 or its complementary sequence,

SEQ ID NO: 12 or its complementary sequence,

SEQ ID NO: 13 or its complementary sequence, and

SEQ ID NO: 14 or its complementary sequence,

and also encompasses any solid surface (or carrier) which comprises the fourteen amplification products obtainable by submitting human genomic DNA (such as e.g. DNA from human lymphocytes collected from a healthy individual) to polymerase chain reactions with each of the following primer pairs:

SEQ ID NO: 15 (or its complementary sequence) and SEQ ID NO: 16 (or its complementary sequence),

SEQ ID NO: 17 (or its complementary sequence) and SEQ ID NO: 18 (or its complementary sequence),

SEQ ID NO: 19 (or its complementary sequence) and SEQ ID NO: 20 (or its complementary sequence),

SEQ ID NO: 21 (or its complementary sequence) and SEQ ID NO: 22 (or its complementary sequence),

SEQ ID NO: 23 (or its complementary sequence) and SEQ ID NO: 24 (or its complementary sequence),

SEQ ID NO: 25 (or its complementary sequence) and SEQ ID NO: 26 (or its complementary sequence),

SEQ ID NO: 27 (or its complementary sequence) and SEQ ID NO: 28 (or its complementary sequence),

SEQ ID NO: 29 (or its complementary sequence) and SEQ ID NO: 30 (or its complementary sequence),

SEQ ID NO: 31 (or its complementary sequence) and SEQ ID NO: 32 (or its complementary sequence),

SEQ ID NO: 33 (or its complementary sequence) and SEQ ID NO: 34 (or its complementary sequence),

SEQ ID NO: 35 (or its complementary sequence) and SEQ ID NO: 36 (or its complementary sequence),

SEQ ID NO: 37 (or its complementary sequence) and SEQ ID NO: 38 (or its complementary sequence),

SEQ ID NO: 39 (or its complementary sequence) and SEQ ID NO: 40 (or its complementary sequence),

SEQ ID NO: 41 (or its complementary sequence) and SEQ ID NO: 42 (or its complementary sequence).

Examples of appropriate solid surface or carrier include any DNA-appropriate filters such as nylon® membranes (see e.g. EP 1 098 004 in the name of Fuji Photo Film Co. Ltd “Fixation of nucleotide derivatives to solid carrier”), and any DNA arrays or micro-arrays such as DNA chips. DNA arrays or micro-arrays such as DNA chips can be produced following any standard procedure in the field, this notably includes the procedure and material described in Gasch et al. 2000 (Molecular Biology of the Cell, vol. 11, pages 4241-4257), in U.S. Pat. No. 5,744,305 in the name of Fodor et al., U.S. Pat. No. 5,800,992 in the name of Fodor et al., U.S. Pat. No. 5,807,522 in the name of Brown et al., U.S. Pat. No. 5,552,270 in the name of Khrapku, U.S. Pat. No. 5,736,257 in the name of Conrad et al., U.S. Pat. No. 5,847,019 in the name of Conrad et al., U.S. Pat. No. 6,174,683 in the name of Hahn et al., EP 1 094 318 in the name of NGK Insulators, Ltd., WO 01/26799 in the name of Bio-Informatics Group, Inc.

The products of the invention can be comprised in a biotechnology kit, -e.g. a kit for research in biology, or a kit for drug screening, or a kit for determining the evolution of a pathological disease. They can notably be comprised in a kit for determining CYP450 expression pattern (CYP450 mRNA levels). The present application thus encompasses a kit for determining CYP450 mRNA levels in a biological sample, which comprises:

at least one product selected from the group consisting of the polynucleotides of the invention, the primer pairs of the invention, the amplification products of the invention, the transfection vectors of the invention, the genetically engineered cells of the invention, the solid surfaces of the invention,

optionally further comprising a detection label, and/or a biotechnology-appropriate buffer (e.g. a phosphate buffer).

The products of the invention are very valuable tools for any application wherein CYP450 detection is involved. Indeed, the probes and primers of the invention enable direct detection of CYP450 DNA/cDNA/RNA by hybridation therewith or, respectively, by amplification thereof. According to an advantageous feature, they allow the simultaneous detection of the main fourteen human liver CYP450 isoforms, and this single-step process further require less input biological material than prior art multi-step procedures.

The present application thus relates to a method for the detection of at least one CYP450 isoform selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, in a biological sample which contains polynucleotides, wherein said CYP450 isoform is detected by detection of the presence or absence of its DNA or RNA in said biological sample. This can be achieved

either by hybridization of total DNA or reverse-transcribed total RNA of said sample with at least one polynucleotide of the invention, or with at least one amplification product of the invention, or with a solid surface according to the invention,

or by amplification performed on said total DNA or reverse-transcribed total RNA with at least one primer pair of the invention.

Any condition the skilled person finds favorable to hybridization or, respectively amplification, is appropriate. Appropriate hybridization, or respectively amplification conditions are known to the skilled person and, if desired, they can be adjusted for each particular case by routine standard procedures. Examples of preferred amplification and hybridization conditions are given in the examples below.

Advantageously, the invention provides a method for the simultaneous detection of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, in a biological sample which contains polynucleotides, wherein said CYP450 isoforms are detected by detection of the presence or absence of their respective DNA or RNA in said biological sample. This can be achieved by hybridization of total DNA or reverse-transcribed total RNA of said sample with all SEQ ID NO: 1-14 probes of the invention (or their complementary sequences), and preferably with a solid surface according to the invention (a positive hybridization signal implying presence of the corresponding CYP450, a negative signal implying its absence). Alternatively, this detection can be achieved by PCR or RT-PCR amplification (e.g. multi-well PCR/RT-PCR, preferably multi-well quantitative PCR/RT-PCR) with SEQ ID NO: 15-16 to 41-42 primer pairs (or their complementary sequences)—actual amplification implying CYP450 presence, no amplified product implying CYP450 absence-. Appropriate hybridization or amplification conditions are as above-mentioned.

More particularly, the present application relates to a method for measuring the mRNA level there is in a biological sample for a CYP450 selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:

total RNA of said biological sample is reverse transcribed, and the cDNA thus obtained is placed into contact under conditions appropriate for DNA/DNA hybridizations with at least one polynucleotide of the invention, or with at least one amplification product of the invention, or with a solid surface according to any one of the invention, or under conditions appropriate for DNA amplification with at least one primer pair of the invention, and wherein

the intensity of the hybridization or amplification signal which is thus obtained is measured,

said CYP450 mRNA level corresponding to this measured intensity.

According to a further advantageous aspect, the present invention provides a method for simultaneously measuring the mRNA levels there are in a biological sample for CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:

total RNA of said biological sample is reverse transcribed, and the cDNA thus obtained is placed into contact under conditions appropriate for DNA/DNA hybridizations with all SEQ ID NO: 1-14 probes of the invention (or their complementary sequences), and preferably with a solid surface according to the invention, and wherein

the respective hybridization signals thus obtained are measured, said CYP450 mRNA levels respectively corresponding to these measured hybridization signals.

Alternatively, said cDNA can be placed under conditions appropriate for DNA amplification with every one primer pair of the invention, i.e. SEQ ID NO: 15-16 to 41-42 or their complementary sequences (e.g. on a multi-well PCR/RT-PCR, preferably multi-well quantitative PCR/RT-PCR), and the respective amplification signals thus obtained are measured, said CYP450 mRNA levels respectively corresponding to these measured amplification signals.

Appropriate DNA/DNA hybridization or PCR/RT-PCT conditions are as above-mentioned.

The present application also encompasses any method for evaluating the influence a compound has on the mRNA level(s) there is(are) in a biological sample for one or several CYP450 DNA selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:

the mRNA levels of this or these CYP450 in the presence of said compound and in the absence of said same compound are measured by a method for mRNA measurement of the invention, and wherein

the influence of said compound is evaluated by comparison of the two mRNA levels thus measured.

This method for evaluating the influence a compound has on the mRNA level(s) of said one or several CYP450 can advantageously be used:

as a method for screening for safer and/or more efficient drugs among a set of drug candidates, which takes into account the influence each drug candidate has on CYP450 mRNA levels, and/or

as a method for evaluating the metabolization of a compound in a living organism which takes into account the influence said compound may have on CYP450 mRNA levels.

The present application also relates to a method for determining the effect a compound may have when administered to a living organism expressing at least one CYP450 isoform selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, or for determining interactions between compounds intended for administration to a living organism expressing at least one of such a CYP450 isoform, wherein:

the influence which each of said compound(s) has on the mRNA level of said at least one CYP450 isoform is evaluated by said method for evaluating the influence a compound has on the mRNA level(s) there is(are) in a biological sample for said one or several CYP450, and wherein

the effect it may have (or the interactions they may induce) in said living organism is (are) evaluated by comparison of their respective influences.

To implement the methods of the invention, any biological sample containing polynucleotides is appropriate. Preferred biological samples notably comprise total reverse-transcribed RNA from human hepatocytes.

The invention is illustrated by the following examples and accompanying drawings.

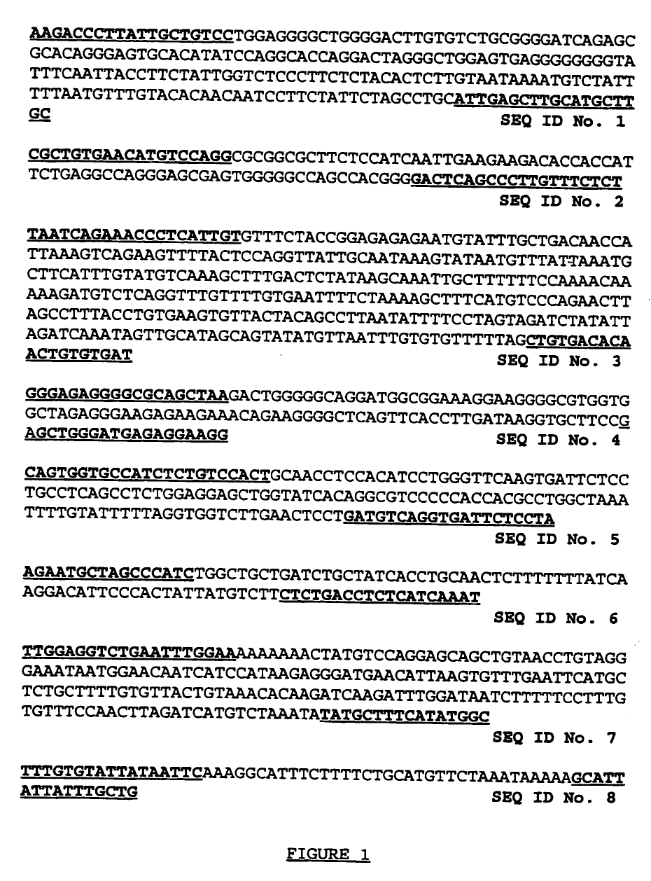

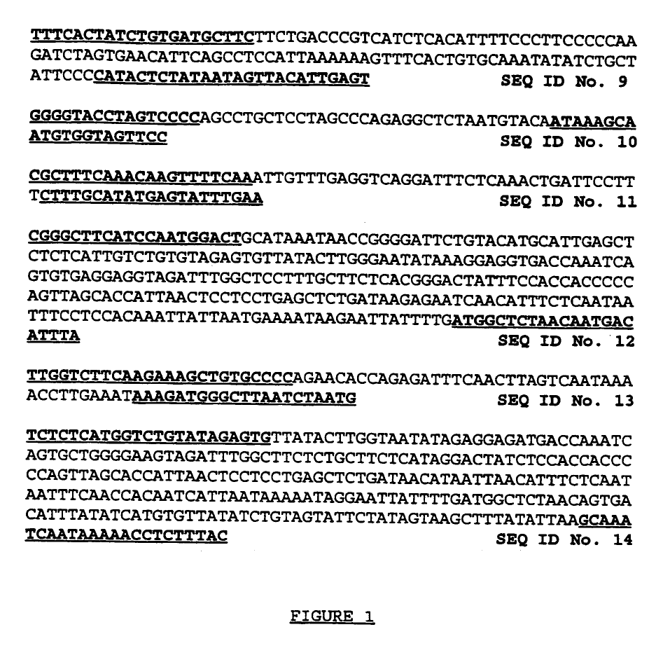

FIG. 1 gives SEQ ID NO: 1-14 sequences (cDNA probes).

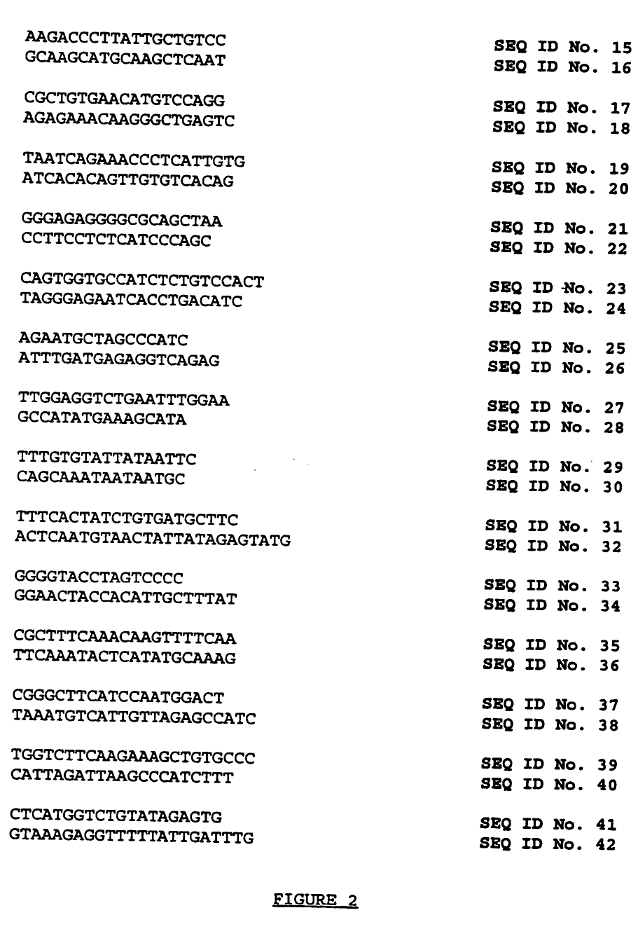

FIG. 2 gives SEQ ID NO: 15-16 to 41-42 sequences (primer pairs specific to SEQ ID NO: 1-14).

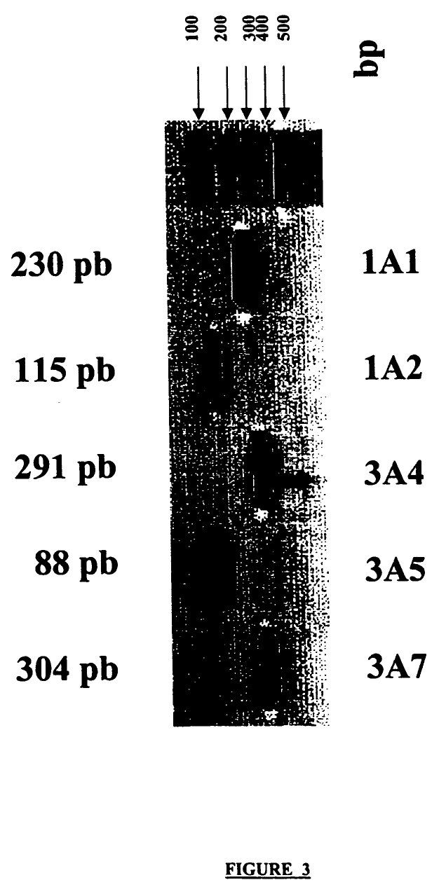

FIG. 3 is an agarose gel illustrating with CYP 1A1, CYP 1A2, CYP 3A4, CYP 3A5, CYP 3A7 that the primers of the invention amplify one single product of the expected size.

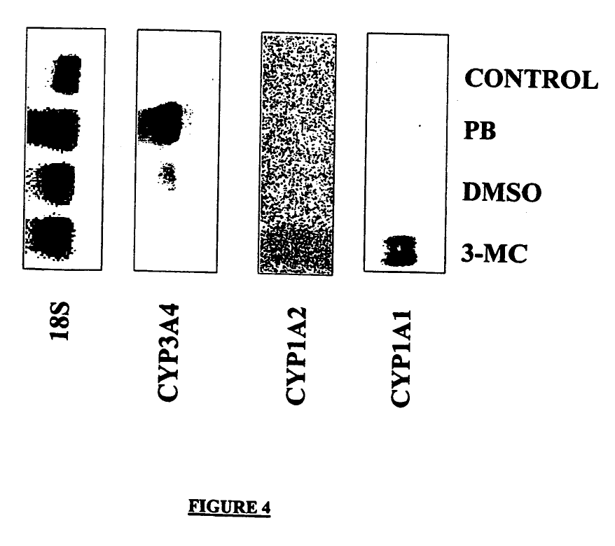

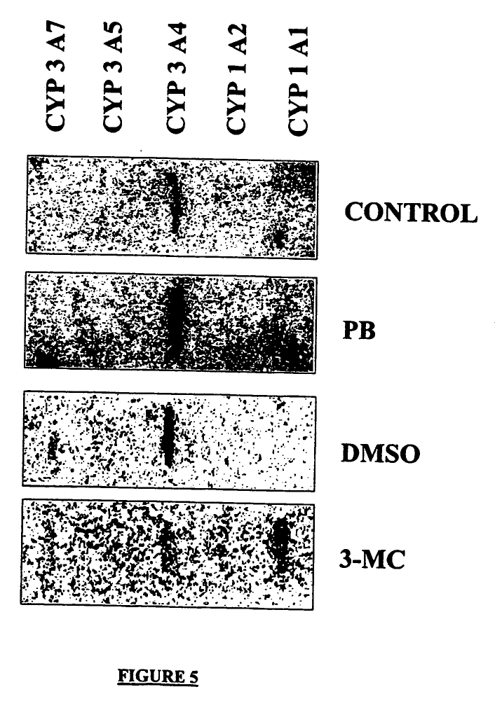

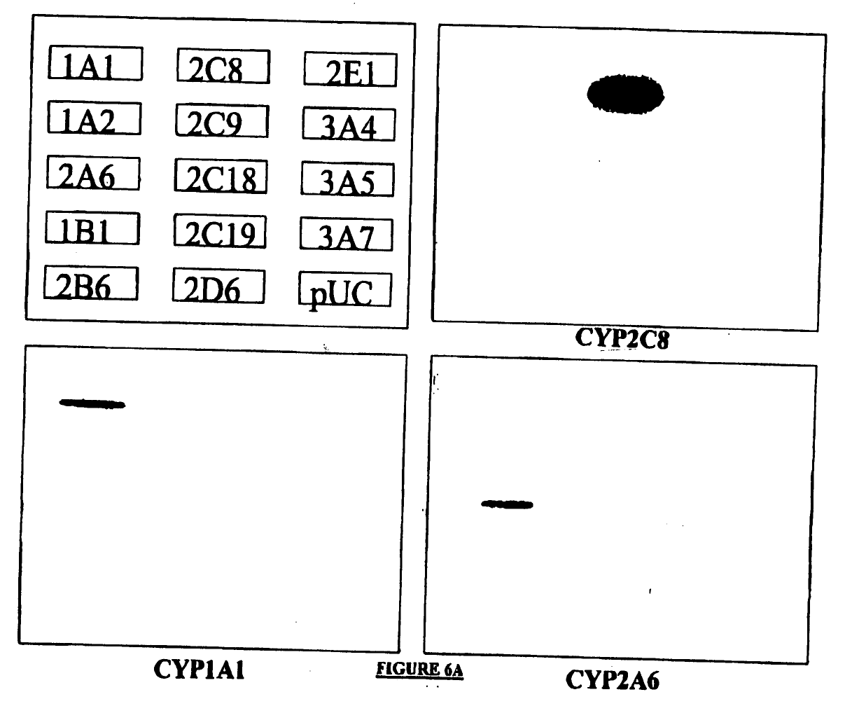

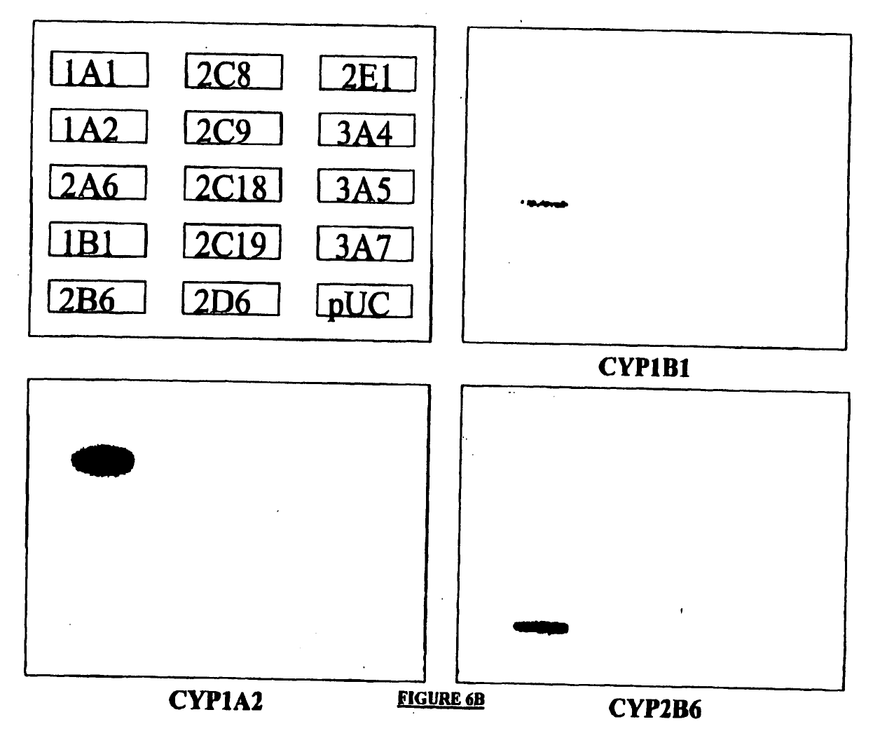

FIG. 4 shows representative Northern blots obtained for CYP 1A1, CYP 1A2 and CYP 3A4 hybridizations (18S as a reference) with total RT-RNA from human hepatocytes (first lane: control, without treatment; second lane: PB treatment; third lane: DMSO treatment; fourth lane: 3-MC treatment).

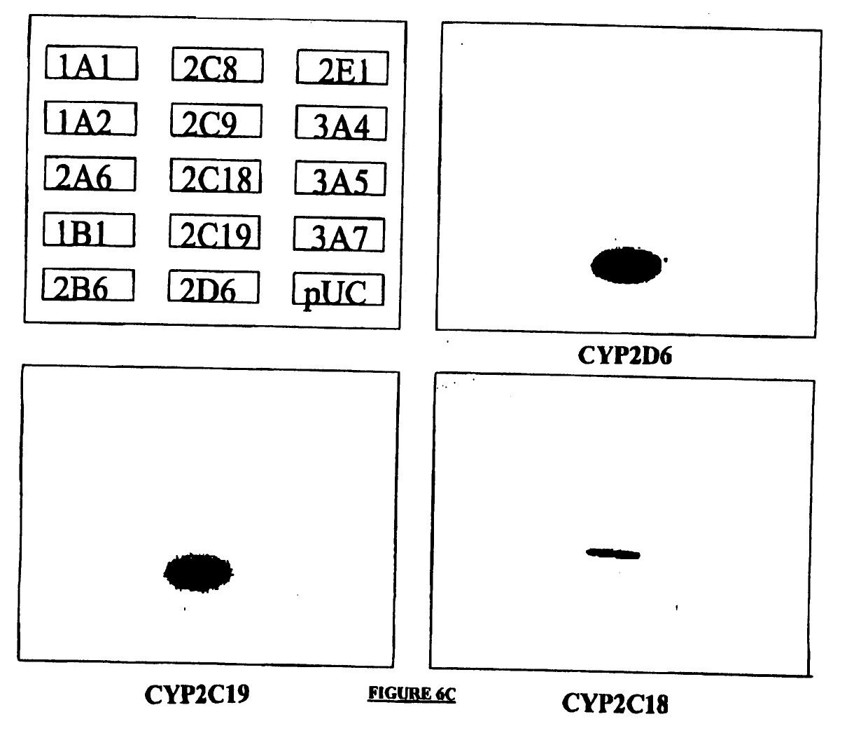

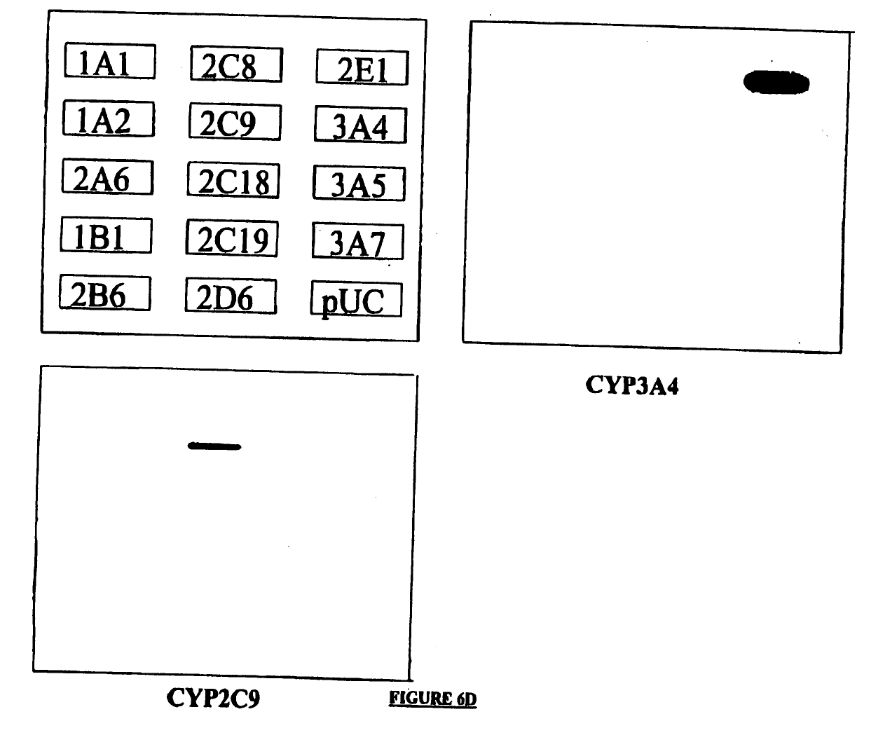

FIG. 5 shows a representative slot blot obtained by hybridization of total RT-RNA from human hepatocytes (first lane: control, without treatment; second lane: PB treatment; third lane: DMSO treatment; fourth lane: 3-MC treatment) with a membrane of the invention which comprises the cDNA probes specific to CYP 1A1 (SEQ ID NO: 1), CYP 1A (SEQ ID NO: 2), CYP 3A4 (SEQ ID NO: 12), CYP 3A5 (SEQ ID NO: 13), CYP 3A7 (SEQ ID NO: 14).

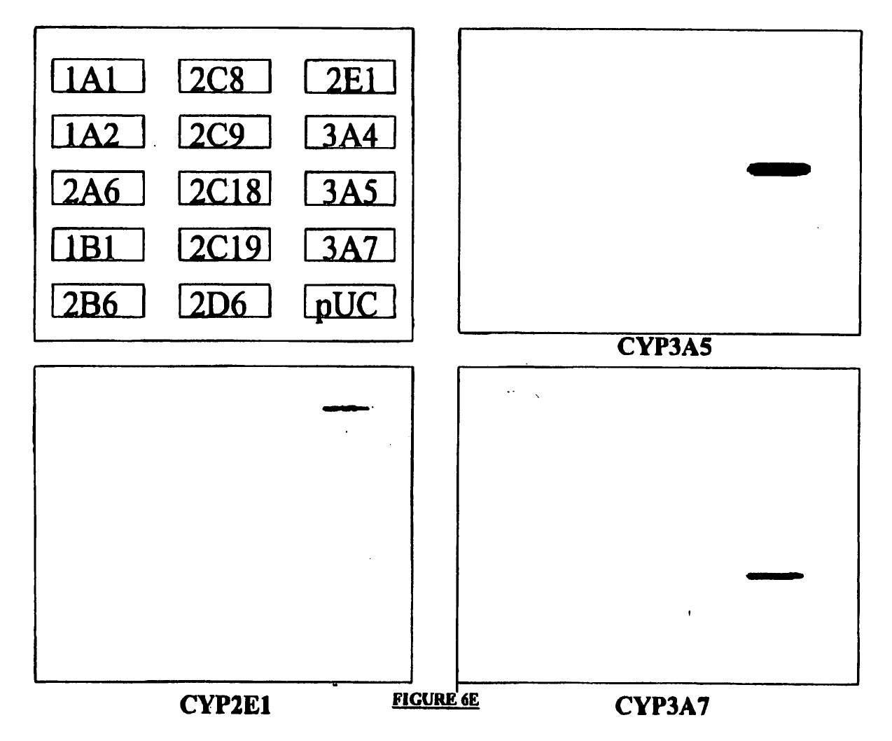

FIGS. 6A-6E illustrate representative results obtained when hybridizing a probe of the invention with a nylon® membrane onto which have deposited 50 nanograms of each fourteen plasmidic DNA, and of empty plasmid (total=14 membranes): the name appearing at the bottom of each membrane indicates which probes has been used for this membrane, in the upper left corner of each FIG. 6 drawing sheet, is represented the location of each inserted plasmidic DNA (name of the inserted probe), and of the empty plasmid (pUC).

### EXAMPLE 1

### Amplification and Cloning

Isolation of the cDNA Probes with the Primers—One Single Band of the Expected Size is Obtained

Standard PCR conditions have been used to amplify the cDNA probes of SEQ ID NO: 1-14 with the primer pairs of SEQ ID NO: 15-16 to 41-42. The final concentrations in dNTPs, Taq Polymerase Cetus® (Perkin Elmer), and of each of said respective primers were 1.25 mM, 2.5IU and 0.4 mM. Amplifications were performed with human genomic DNA (DNA from human lymphocytes collected from healthy individuals). MgCl2 content, amplification temperature and the expected fragment size are given in the table 2 below.

TABLE 2

| | | | MgCl2 |
|---|---|---|---|
| CYP450 | Expected fragment | | Concentration |
| isoform | size | PCR temperature | (mM) |
| 1A1 | 230 | 55 | 1.5 |
| 1A2 | 115 | 55 | 3 |
| 1B1 | 352 | 52 | 1.5 |
| 2A | 133 | TD65-55 and 55 | 2.5 |
| 2B6 | 164 | 51 | 2 |
| 2C8 | 100 | 50 | 2.5 |
| 2C18 | 215 | 51 | 2 |
| 2C9 | 68 | 48 | 2.5 |
| 2C19 | 146 | 52 | 1.5 |
| 2D6 | 71 | 55 | 1.5 |
| 2E1 | 82 | 52 | 3 |
| 3A4 | 291 | 55 | 1.5 |
| 3A5 | 88 | TD65-55 and 55 | 3 |
| 3A7 | 304 | 52 | 2 |

TD = Touch down PCR (0.5° C. degree decrease per cycle every 30 seconds),
i.e.:
94° C. for 5 min
20 cycles 94° C. for 30 sec
65° C. to 55° C. [−0.5° C. per cycle every 30 sec]
72° C. for 30 sec
25 cycles 94° C. for 30 sec
55° C. for 30 sec
72° C. for 30 sec
72° C. for 30 sec
For all other PCRs:
94° C. for 5 min
45 cycles 94° C. for 30 sec
PCR temperature for 30 sec
72° C. for 30 sec
72° C. for 30 sec

Five microliters of amplification product have been deposited onto a 2% agarose gel, and submitted to standard electrophoresis. UV visualization shows only one band for each amplification product, and each band was of the expected size. FIG. 3 illustrates the results thus obtained with CYP 1A1, CYP 1A2, CYP 3A4, CYP 3A5 and CYP 3A7.

cDNA Purification, Cloning and Sequencing:

The complementary DNA obtained by PCR were purified with the QLAquick™ PCT purification kit (Qiagen®, Courtaboeuf, France) so as to eliminate excess nucleotides, primers, enzymes and salts. The thus purified cDNA were then digested with EcoR1.

Twenty micrograms of pUC 19 (Genbank X02415) were digested by EcoR1 in 20 microliters of water, and were 5′-dephosphorylated by 20 IU of alkaline phosphatase (Biolabs® Ozyme, St Quentin en Yvelines, France) at 37° C. for 1 hour. The reaction is stopped by addition of 5 mM of EDTA (250 mM, pH 8) and heating at 65° C. for 15 min.

The DNA to be inserted and 800 IU of T4 DNA ligase (Biolabs®) are added to the linearized and dephosphorylated vector. Incubation is conducted at 16° C. for at least 12 hours. The plasmid is preferably at a low concentration by comparison to the DNA to be inserted (such as e.g. 1:5 ratio in molar concentrations).

Fifteen microliters of competent E. coli XL1 Blue were transformed by thermal shock with 20 microliters of ligation product at 42° C. for 1 min. Ampicillin-resistant colonies were selected from LB-agar culture medium comprising the antibiotic after an overnight culture at 37° C. The thus selected colonies were overnight incubated in 2 milliliters of LBA medium at 37° C. under constant shaking.

Actual DNA insertion is checked by DNA extraction (QIAprep Spin Miniprep Kit, Qiagen®), digested by EcoR1, and then deposited onto a 2% agarose gel. Positive clones (which comprise the insert) are then placed under culture again. Plasmidic DNA is then extracted with Maxiprep, Qiagen® plasmid maxi kit, and sequenced (AB Prism 310 genetic analyser, Perkin Elmer®). Sequencing is performed with primers which hybridize to pUC plasmid and frame the cloning sites, following modified Sanger method with fluorochrome-labeled oligonucleotides (Sanger F, Nickeln S and Coulson A R, 1992, DNA sequencing with chain-terminating inhibitors, Biotechnology 24: 104-108).

The sequences thus obtained correspond to the claimed SEQ ID NO: 1-14.

### EXAMPLE 2

### Specificity of the Amplified cDNA

Production of a Solid Surface Comprising Either the Amplification Products or the the cDNA Probes:

On nylon® membrane filters, the following DNA sequences were deposited:

either the sequences obtained by amplification of 50 nanograms of plasmidic DNA as described in the above example 1, followed by purification and UV spectrophotometry dosage (Power Wave X2, Biotek instruments®, St Quentin en Yvelines, France),

or plasmidic DNA (50 nanograms of linearized plasmid containing a probe inserted therein, or of empty pUC plasmid) obtained from the clones described in the above example 1 after linearization by Bam H1 (20 IU for 20 micrograms of plasmidic DNA, 1 hour at 37° C.).

The amplified sequences as well as the plasmidic ones were denatured by EDTA (final concentration of 10 mM, 10 min at 65° C.) and then by NaOH (final concentration of 0.1 M, 30 min at ambient temperature), and neutralized by SSC6× buffer, before being deposited onto a nylon® membrane (Biosupport®, Pall, Portsmouth, UK). Binding to the membrane was achieved by exposure to UV for 4 min and heating at 80° C. for 2 hours.

Specificity Assays:

Three types of specificity assays have been performed:

intra-probe specificity: each cDNA probe (SEQ ID NO: 1-14) has been labeled with ³²P following Megaprime® DNA Labelling System (Amersham Pharmacia Biotech, Elancourt, France) and according to random primer polymerization. Each radio-active probe is then incubated for hybridization with one of the above-mentioned filters (2 hours at 65° C. in a Amersham Rapid-Hybrid® buffer—hybridization oven Appligene®—). The filters are then washed at 65° C. for 30 min with SSC 2×, SDS 0.1%, and then with SSC 0.5× SDS 0.1% and last with SSC 0.1× SDS 0.1% (SSC 20× has a pH of 7, and a composition of 3M sodium chloride and 0.3M sodium citrate; SDS 10% is made of sodium dodecyl sulfate salt at 10 g/100 ml diluted in hot water). The filters are then exposed in a phosphorimager cassette (Storm®, 840).

specificity with respect to total RNA from human he atocytes: the same probes have been hybridized to a Northern blot onto which 10 micrograms of total RNA from human hepatocytes have deposited.

specificity with respect to the 3′-NTR and coding regons of the CYP450 cDNA: filters comprising the whole coding sequence or the whole 3′NTR regions of the fourteen CYP450 isoforms have been prepared similarly to what has been above described for probe filters, and each probe has been incubated for hybridization with these filters.

Results:

Each probe except SEQ ID NO: 9 (Le. the CYP 2C19 probe) only hybridizes with its target: there is no cross-hybridization with another CYP450 isoform, nor with the empty plasmid that has been used for transfection (see FIGS. 6A-6E for an illustration of the results), nor with any other RNA from human hepatocytes (as evaluated by Northern blots).

Each probe hybridizes with its target cDNA without cross-hybridizing with another probe. But, where it is clear that the probes for family 3 do not hybridize to 3′-NTR of a non-target family 3 CYP450, it cannot be excluded that SEQ ID NO: 9 may hybridize in the 3′-NTR region of CYP 2C9 (in addition to its CYP 2C19 target). But, SEQ ID NO: 8 only hybridizes to its CYP 2C9 target.

No probe hybridizes with a coding region of the CYP450 isoforms.

SEQ ID NO: 1-14 and their complementary sequences therefore represent a set of cDNA probes which has full specificity with respect to the main fourteen human CYP450 isoforms whose expression is altered by xenobiotics (human liver isoforms).

### EXAMPLE 3

### Membrane Assays (Validation with Xenobiotics known to Induce Regulation of CYP450 Expression)

Human Hepatocyte Culture and Treatment:

Human hepatocytes from healthy individuals originating from cultures in 24-well plaques, at a ratio of 300,000 cells per well without antibiotics nor fungicide have been submitted to the following treatments:

TABLE 3

| | Final | Exposure | Dis- | CYP450 |
|---|---|---|---|---|
| Treatment | molarity | duration | solution | induced |
| 3-methylcholanthrene | 5 microM | 48 hours | DMSO | CYP 1A |
| (3-MC) | | | | |
| Phenobarbital (PB) | 1 microM | 48 hours | Culture | CYP 3A |
| | | | medium | |
| Rifampicine (RIF) | 50 microM | 48 hours | DMSO | CYP 3A |

3-CM control: DMSO, final molarity 0.50% exposure, duration 48 hours. Culture medium has been replaced at t = 24 hours. The quantity of DMSO per well does not exceed 0.5% (final concentration).

RNA Isolation:

600 microliters of solution D (pH7; guanadium thiocyanate 4M; sodium citrate 25 mM; sarcosyl 0.5%; and 1.4 ml of beta-mercaptoethanol per 200 ml of solution D) have been added to each well so as to induce cell lysis. Cell lysates have then been collected and group together as per treatment. Are then successively added: 0.1 volume of sodium acetate (2M, pH4), 1 volume of water-saturated phenol and 0.2 volume of chloroform/isoamylalcohol (49/1, V/V). At each step, the suspension is vigorously vortexed, then left to cool on ice for 15-20 min, and centrifigated for 30 min at 4000 g and 4° C. An equal volume of isopropanol is added after removal of the aqueous phase.

RNA are then precipitated (1 hour at −20° C.) and centrifuged for 30 min at 4000 g and 4° C. The supernatant is removed and the pellets are re-suspended in 2.7 ml of solution D. 2.7 ml of isopropanol are then added to the suspension, and the RNA are again precipitated (1 hour at −20° C.) and centrifugated. The pellets are washed with 5.4 ml of 70% ethanol. After a final centrifugation and removal of the supernatant, the pellets are air-dried, and re-suspended in sterile water containing DEPC (DEPC 1/1000 in water, then sterilization). Total RNA contents are measured on a UV spectrophotometer.

Reverse Transcription of Total RNA:

25 micrograms of total RNA have been placed into contact with oligo-dT (0.4 mM final of an equimolar composition of oligod(T) and of three oligosd(T) which have an A or a G or a C in 3′), and heated to 70° C. for 10 min, then incubated for 5 min on ice so as to allow oligo-dT hybridization on polyA RNA. The four nucleotides have then been added (DATP, dGTP, dCTP, dTTP—0.5 mM final for each), as well as 1.1 U/ml of ribonuclease inhibitor, radio-labeled nucleotides (dATP* and dTTP*; 3,000 Ci/mmol, 10 microCi/microliter—0.66 microCi/microliter in final), MMLV-RT (6 U/microliter final) and its buffer (1× final). The whole mix is incubated at 37° C. for 1 hour, then the cDNA thus formed are denatured by 2.5M NaOH for 10 min at 37° C., and neutralized with Hepes buffer (1M—pH 8). The labeled cDNA thus produced are then purified. An aliquot is deposited onto an acrylamide gel so as to check the quality of the reverse transcription.

In parallel, the membranes are incubated with a pre-hybridization buffer (de-ionized formamide 50%, SDS 2.3%, salmon sperm DNA 0.2 mg/ml, tRNA 0.1 mg/ml, Denhardt 10×, SSEP 3×—SSPE 20× (pH 7.4)=NaCl 3M+NaH₂PO₄0.2M+EDTA 25 mM—) into which salmon sperm DNA has been added (1.0 mg/ml, Salmon testes DNA for hybridization, Sigma) so as to hybridize the non-specific sites, for 24 hours at 42° C.

The labeled cDNA are then hybridized with the filters (48 hours at 42° C.). So as to compare the treatment results with the control results, the different hybridizations have been adjusted with respect to the number of counts per minute (about 400,000 cpm) measured by a liquid scintillation analyzer (Tri Carb 2100 TR, Packard®, Rungis, France). Filters have then been washed with SSC 2× buffer without SDS for 30 min at 42° C., and exposed overnight in a phosphorimager cassette.

For comparison purposes, Northern blots comprising 10 micrograms of total RNA have been incubated for hybridization with each cDNA probe separately. Quantifications are achieved by measuring band intensities per 18S quantity (as evaluated by ethidium bromide coloration).

Results:

The results obtained with the solid surfaces of the invention (membranes comprising the whole set of cDNA probes) are similar to those obtained with one Northern blot per probe. These results are illustrated by FIGS. 4 and 5 wherein Northern blots obtained for CYP 1A1, CYP 1A2, CYP 3A4 (shown in FIG. 4) can be compared to the slot blot obtained with the membrane of the invention (FIG. 5).

In the particular case of the present assays, it can be observed that 3-methylcholanthrene induces an increase in CYP 1A1, and also but more weakly in CYP 1A2, whereas it induces a decrease in CYP 3A4. The phenobarbital and rifampicine treatments induces a strong decrease in CYP 3A4, whereas no variation is observed for CYP 14 sub-family and for CYP 3A5 and 3A7. Quantification of the hybridization signals are of the same order in Northern blots compared to slot blot according to the invention.

The membranes of the invention advantageously require less biological material than the Northern blot technique, and this is a very useful feature when hepatic biopsies and culture are to be performed. They are also less time-consuming as only one hybridization gives the full results (instead of 14 Northern blots).

Should it be desired to further enhance the sensitivity of the method according to the invention, DNA micro-arrays can then advantageously be used instead of the above-described nylon® membranes.

All publications, patents, patent applications cited in this specification are herein incorporated by reference as if each individual publication, patent, patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit and scope of the appended claims.

1. Isolated and purified polynucleotide selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, and the complementary sequences thereof.

2. Prime pair selected from the group consisting of
SEQ ID NO: 15 and SEQ ID NO: 16,
SEQ ID NO: 17 and SEQ ID NO: 18,
SEQ ID NO: 19 and SEQ ID NO: 20,
SEQ ID NO: 21 and SEQ ID NO: 22,
SEQ ID NO: 23 and SEQ ID NO: 24,
SEQ ID NO: 25 and SEQ ID NO: 26,
SEQ ID NO: 27 and SEQ ID NO: 28,
SEQ ID NO: 29 and SEQ ID NO: 30,
SEQ ID NO: 31 and SEQ ID NO: 32,
SEQ ID NO: 33 and SEQ ID NO: 34,
SEQ ID NO: 35 and SEQ ID NO: 36,
SEQ ID NO: 37 and SEQ ID NO: 38,
SEQ ID NO: 39 and SEQ ID NO: 40,
SEQ ID NO: 41 and SEQ ID NO: 42 and
their respective complementary oligonucleotide couples.

3. Process for producing a CYP450-specific probe, selected from the group consisting of CYP 1A1, CYP 1A2, CYP 1B1, CYP 2A, CYP 2B6, CYP 2C8, CYP 2C18, CYP 2C9, CYP 2C19, CYP2D6, CYP 2E1, CYP 3A4, CYP 3A5, GYP 3A7, characterised in that human genomic DNA or human reverse-transcribed GYP450-containing RNA is submitted to PCR with a primer of claim 2, whereby the amplification product thus generated corresponds to said CYP450-specific probe.

4. Transfection vector, in which the vector insert consists of a polynucleotide of claim 1.

5. Genetically engineered cell, which has been transfected with a polynucleotide of claim 1, or with a transfection vector of claim 4.

6. The genetically engineered cell of claim 5, wherein an oligonucleotide of claim 1 replicates when the cell is made to grow.

7. Solid surface which comprises at least one oligonucleotide of claim 1, or at least one transfection vector of claim 4, or at least one genetically engineered cell of claim 5 or 6.

8. The solid surface of claim 7, which comprises:
SEQ ID NO: 1 or its complementary sequence,
SEQ ID NO: 2 or its complementary sequence,
SEQ ID NO: 3 or its complementary sequence,
SEQ ID NO: 4 or its complementary sequence,
SEQ ID NO: 5 or its complementary sequence,
SEQ ID NO: 6 or its complementary sequence,
SEQ ID NO: 7 or its complementary sequence,
SEQ ID NO: 8 or its complementary sequence,
SEQ ED NO: 9 or its complementary sequence,
SEQ ED NO: 10 or its complementary sequence,
SEQ ID NO: 11 or its complementary sequence,
SEQ ID NO: 12 or its complementary sequence,
SEQ ED NO: 13 or its complementary sequence, and
SEQ ID NO: 14 or its complementary sequence.

9. The solid surface of claim 7, which comprises the fourteen amplification products obtainable by submitting human genomic DNA to polymerase chain reactions with each of the following primer pairs:
SEQ ID NO: 15 (or its complementary sequence) and SEQ ID NO: 16 (or its complementary sequence),
SEQ ID NO: 17 (or its complementary sequence) and SEQ ID NO: 18 (or its complementary sequence),
SEQ ID NO: 19 (or its complementary sequence) and SEQ ID NO: 20 (or its complementary sequence),
SEQ ID NO: 21 (or its complementary sequence) and SEQ ID NO: 22 (or its complementary sequence),
SEQ ID NO: 23 (or its complementary sequence) and SEQ ID NO: 24 (or its complementary sequence),
SEQ ID NO: 25. (or its complementary sequence) and SEQ ID NO: 26 (or its complementary sequence),
SEQ ID NO: 27 (or its complementary sequence) and SEQ ID NO: 28 (or its complementary sequence),
SEQ ID NO: 29 (or its complementary sequence) and SEQ ID NO: 30 (or its complementary sequence),
SEQ ID NO: 31 (or its complementary sequence) and SEQ ID NO: 32 (or its complementary sequence),
SEQ ID NO: 33 (or its complementary sequence) and SEQ ID NO: 34 (or its complementary sequence),
SEQ ID NO: 35 (or its complementary sequence) and SEQ ID NO: 36 (or its complementary sequence),
SEQ ID NO: 37 (or its complementary sequence) and SEQ ID NO: 38 (or its complementary sequence),
SEQ ID NO: 39 (or its complementary sequence) and SEQ ID NO: 40 (or its complementary sequence),
SEQ ID NO: 41 (or its complementary sequence) and SEQ ID NO: 42 (or its complementary sequence).

10. The solid surface according to any one of claims 7-9, which is selected from the group consisting of the DNA-appropriate filters such as nylon® membranes, and the DNA arrays or micro-arrays such as the DNA chips.

11. Kit for determining CYP450m RNA levels, which comprises:
at least one product selected from the group consisting of the polynucleotides of claim 1, the primer pairs of claim 2, the transfection vectors of claim 4, the genetically engineered cells of claims 5 and 6, the solid surfaces of claims 7, 8, 9 and 10,
optionally further comprising a detection label, and/or a biotechnology-appropriate buffer.

12. A method for the detection of at least one CYP450 isoform selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, in a biological sample which contains polynucleotides, wherein said CYP450 isoform is detected
either by hybridization of total DNA or reverse-transcribed total RNA of said sample with at least one polynucleotide of claim 1, or with a solid surface according to any one of claims 7-9,
or by amplification performed on said total DNA or reverse-transcribed total RNA with at least one primer pair of claim 2.

13. A method for the simultaneous detection of CYP1A1, CYP 1A2, CYP 1B1, CYP 2, CYP 2B6, CYP 2C8, CYP 2C18, CYP 2C9, CYP 2C19, CYP 2D6, CYP 2E1, CYP 3A4, CYP 3A5 and CYP 3A7, in a biological sample which contains polynucleotides, wherein said CYP450 isoforms are detected by hybridization of total DNA or reverse-transcribed RNA of said sample with a solid surface according to any one of claims 8-9, or by DNA or cDNA amplification with SEQ ID 15-16 to 41-42 primer pair of claim 2 (or their complementary sequences).

14. A method for measuring the mRNA level there is in a biological sample for a CYP450 selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:
total RNA of said biological sample is reverse transcribed, and the cDNA thus obtained is placed into contact under conditions appropriate for DNA/DNA hybridizations with at least one polynucleotide of claim 1, or with a solid surface according to any one of claims 7-9, or under conditions appropriate for DNA amplification with at least one primer pair of claim 2, and wherein
the intensity of the hybridiztion or amplification signal which is thus obtained is measured,
said CYP450 mRNA level corresponding to this measured intensity.

15. A method for simultaneously measuring the mRNA levels there are in a biological sample for CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:
total RNA of said biological sample is reverse transcribed, and the cDNA thus obtained is placed into contact under conditions appropriate for DNA hybridizations with a solid surface according to any one of claims 8-9, or under conditions appropriate for cDNA amplification with SEQ ID NO: 15-16 to 41-42 primer pairs of claim 2 (or their complementary sequences), and wherein
the respective hybridization or amplification signals thus obtained are measured,
said CYP450 mRNA levels respectively corresponding to these measured hybridization or amplification signals.

16. A method for evaluating the influence a compound has on the mRNA level(s) of one or several CYP450 selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, wherein:
the mRNA levels of this or these CYP450 in the presence of said compound and in the absence of said same compound are measured by the method of claim 14 or 15, and wherein
the influence of said compound is evaluated by comparison of the two mRNA levels thus measured.

17. A method for screening for safer and/or more efficient drugs among a set of drug candidates, which takes into account the influence each drug candidate has on CYP450 mRNA levels, wherein said influence is evaluated following the method of claim 16.

18. A method for evaluating the metabolization of a compound in a living organism which takes into account the influence said compound may have on CYP450 mRNA levels, wherein said evaluation is made following the method of claim 16.

19. A method for determining the effect a compound may have when administered to a living organism expressing at least one CYP450 isoform selected from the group consisting of CYP1A1, CYP 1A2, CYP 1B1, CYP 2A, CYP 2B6, CYP 2C8, CYP 2C18, CYP 2C9, CYP 2C19, CYP 2D6, CYP 2E1, CYP 3A4, CYP 3A5 and CYP 3A7, or for determining interactions between compounds intended for administration to a living organism expressing at least one of such a CYP450 isoform, wherein:
the influence each of said compound(s) has on the mRNA level of said at least one CYP450 isoform is evaluated by the method of claim 16, and wherein
the effect it may have (or the interactions they may induce) in said living organism is (are) evaluated by comparison of their respective influences.
